(19) United States
(12) Patent Application Publication
(10) Pub. No.: US 2026/0062659 A1
(43) Pub. Date: Mar. 5, 2026

(54) DYNAMIC SURFACE TENSION SUPPORTED 3-D CELL CULTURE TECHNOLOGY (FLOAT LAYER CELL CULTURE DEVICES)

(71) Applicant: Aloft Biotechnologies, Alachua, FL (US)

(72) Inventor: Isaac Finger-Baker, Alachua, FL (US)

(21) Appl. No.: 19/293,578

(22) Filed: Aug. 7, 2025

Related U.S. Application Data
(60) Provisional application No. 63/680,502, filed on Aug. 7, 2024.

(51) Int. Cl. C12M 1/12; C12M 1/04; C12M 1/09; C12M 1/34

(52) U.S. Cl. CPC C12M 25/04; C12M 23/24; C12M 23/56; C12M 41/34

(57) ABSTRACT

The subject invention pertains to a novel gas exchanger system and methods for culturing spheroids, comprising a substrate exhibiting hierarchical high aspect ratio surface features, where the surface geometry incorporates small dimple indentations in the surface and even smaller vent holes or channels that penetrate through the surface, and coated with a superhydrophobic material that forms a contiguous stabilized plastronic air-liquid interface from surface tension that resists the culture medium volume's pressure and guides cells to form spheroids, where the interface serves as an oxygen source and does not slow down nutrient diffusion giving better control over the culture environment.

### CROSS-REFERENCE TO RELATED APPLICATION

The present application claims the benefit of U.S. Provisional Application Ser. No. 63/680,502, filed Aug. 7, 2024, the disclosure of which is incorporated herein by reference in its entirety.

### BACKGROUND OF THE INVENTION

Culture of cells from humans and other mammals has been possible for almost 100 years [1]. Three different modes of cell culture are bi-dimensional (2D) adherent culture, on a 2D surface, suspension cell culture with individual cells free floating in stirred media, and three-dimensional (3D) clumps of cells growing in a spherical structure, known as spheroids [2,3]. Culturing cells in a 3D format allows scientists to grow cells in an environment, like that found in the human body. These techniques create a microenvironment where cells can naturally form and grow as a spheroid. Spheroids are small spherical formations of cells grown outside of the body. Spheroids are important for many areas of research to model the behavior of tissues or cancers without the need for an entire organism. In a spheroid, cells connect to one another, like they would in tissues, enabling them to exchange both mechanical and chemical signals—and collectively respond to their surroundings. The three-dimensional arrangement of cells in a spheroid allows for the cells to behave in closer approximation to the way they would behave in their parent organism. Modeling this behavior is essential in many areas of medical research, and spheroid culture is often used [3]. Spheroid cell culture has had a great deal of development over the last 60 years. Recent advances in ultra-low adhesion culture surfaces and microcavity geometries allow for reproducible production and scalability. However, providing a microenvironment that closely mimics the natural habitat of cells growing in tissues has been a significant challenge [4]. Yet, these efforts are well justified since 3D cell culture is a powerful tool for studying the complex interactions between cells and microenvironment.

Several 3D cell culture techniques have been developed and are in use for different applications. The development of low-attachment surface treatments has been a key advancement in this field [5]. When a single cell suspension of attachment-dependent cells are seeded onto a normal culture surface, they quickly anchor themselves to the solid surface, spread out, and adopt an un-natural morphology, growing as a single layer (FIG. 1). This influences several other cellular behaviors. In contrast, when seeded onto a low-attachment surface, cells cannot readily anchor themselves and instead remain unattached until they encounter other cells, ultimately forming spheroids. Adding concave microfeatures to the surface further encourages cells to cluster and helps control spheroid size.

A simple, but effective method is “hanging droplet” cell culture (FIG. 2). Briefly, a small volume of cells suspended in growth media is deposited on a sterile culture surface. The surface is inverted, flipped over, so the volume forms a hanging droplet of liquid due to the surface tension of the media. As the cells settle due to gravity, they collect at the bottom of the droplet near to one another. Without a solid mechanical surface available, the cells attach to one another. The air-liquid boundary formed by the surface tension of the droplet provides a dynamic interface, which does not impose mechanical constraints like a solid surface [4,7]. As a bonus, due to the diffusion path length difference, the local oxygen concentration at the surface of the cell sphere is higher than culture techniques at the bottom of a container. This technique has several drawbacks, but progress has been made with various devices to form and handle the droplets. Although this approach got significantly better at reproducibility and 3D shape uniformity, it lacks scalability due to the difficulty of exchanging the media and tenancy can be easily disrupted.

Another simple technique is to grow an adherent cell line in a flat bottom multi-well plate until they form a monolayer. The monolayer is then dislodged by percussing the plate. The cell-cell-ECM mechanical forces result in a 3-dimensional ball of cells. However, this technique also presents difficulty with uniformity and reproducibility.

StemCell Technologies markets AggreWell™, a microfeature based technology in a microplate format, that solves the number of cells per spheroid uniformity issue (FIG. 3). The culture surface is tiled with inverted square pyramids, either 400 μm or 800 μm in size. A cell suspension is placed in the vessel and centrifuged. The angle of the surfaces in the pyramid well causes the cells to cluster at the bottom or the center of the pyramid shape. However, this approach does not provide an even supply of oxygen to the cell clusters.

Corning's most recent development to address reproducibility and uniformity in the production and maintenance of spheroids is the Elplasia plate (FIG. 4) [5]. This device combines ultra-low attachment surfaces and micro cavity geometries to address factors plaguing 3D cell culture. Cells are seeded on an ultra-low attachment (ULA) surface which has 400 μm round bottom microcavities within each macro well. Like ULA, cell growth results in layer contraction and clump formation. This device improves the clump size and shape uniformity, but clumps are still asymmetrical, experience substantial mechanical interaction with the surface, and are size limited due to diffusion. These devices are innovative and perform well, but are very expensive and still only provide limited scalability.

A classic method of 3D cell culture is cells suspended in Matrigel, a basement membrane extract that self-assemble into spheroids due to cell-cell adhesion and matrix confinement (FIG. 5). It is simple and versatile, suitable for various cell types. This method is typically reserved for organoids composed of more than one cell type. Also, there is a need for differentiation which makes the Matrigel expensive and difficult to handle in a reproducible manner [8]. While the technologies described above may offer practical solutions to improve hydrogel organoid cultivation, Matrigel cultures only provide limited scalability and reproducibility.

The common difficulties among current spheroid culture technologies are size/shape uniformity, unknown influence of mechanical interaction with the surface, method reproducibility, scalability, high hands-on demand and appropriate dissolved gas and nutrient availability. The Very Low Adhesion (VLA) and microcavity technologies can decrease the mechanical interaction of the cells with the vessel material, but can still influence cell behavior in an asymmetrical manner. A result of forming the cell clumps by seed layer extra cellular matrix (ECM) contraction is the non-spherical distribution of the cells in the clump, both in the shape of the clump of cells, but also in the mechanical association with the ECM across the shape of the clump.

A less addressed challenge is the spheroid size and more generally a cell culture density limitation due to diffusion/depletion limitations [9]. Whether inside the human body or in culture, cells require oxygen and nutrients while needing to eliminate carbon dioxide and waste products. In conventional culture, where cells grow as a spread-out monolayer, they have a large surface area exposed to the overlaying culture medium, facilitating gas, nutrient, and metabolite exchange. Even while having prime positioning for mass exchange as a monolayer, nutrient consumption and metabolite production can outpace the diffusion coefficient of solutes transiting the height of the overlaying culture medium and provide a growth limiting microenvironment. Furthermore, in spheroids, where the surface area per cell volume for this exchange is reduced, as spheroid size increases this ratio drops even more. Culture conditions influence how a spheroid grows and how it behaves as a model. In most spheroid production platforms, spheroids are formed and maintained at the bottom of a culture dish, beneath a height of overlaying culture medium, either resting on a solid surface or within a microfeature. Gas exchange of oxygen and carbon dioxide occurs at the culture medium's surface, typically a few millimeters deep. As cells consume oxygen and release carbon dioxide at even higher rate densities than those of monolayer culture, these gases must diffuse the entire distance to the oxygen source. For comparison, almost every cell of a human is located within 50 to 100 μm of a capillary. In culture, the required diffusion distance causes a lag in gas concentration at the spheroid surface, limiting the size and health of spheroids. To balance the frequency of feedings with the gas exchange needs of the culture, the volume of culture medium is kept to a minimum. Additionally, nutrient availability is restricted near the surface of the culture device. When cells rest on a solid surface or within a concave feature, free diffusion is only accessible to one side of the cell. At the solid material surface, physical interactions create an interface boundary where diffusion is slower, further limiting nutrient availability in the immediate culture environment. Diffusion of gasses, nutrients, metabolites and signaling factors can be described with a spherical diffusion equation using accumulation factors for metabolism. This geometry imposes the greatest limitations to the cells residing at the center of the clump. This can eventually cause necrosis of the cells at the core from accumulation of waste and deprivation of nutrients and oxygen.

Recently Ultra-low Adhesion (ULA) surface coatings have become popular as they allow for cell clump/ball formation without intervention (FIG. 5). A suspension of cells is seeded onto a ULA culture surface and allowed to form a monolayer. As in the monolayer release method, the forces between the cells cause contraction of the monolayer into a ball or clump, only without the need to physically detach them. The cells detach from the surface under their own cell-cell-ECM forces once a critical confluence is achieved. However, this approach suffers from uniformity and scalability issues but is now the industry norm.

Hanging droplet 3D Cell Culture and its iterations demonstrate the utility of using the surface tension at an air-liquid interface to confine and support cells during cell agglomeration and growth (FIG. 5). The non-solid, fluid nature of the air-liquid interface provides a dynamic boundary, to which the cells cannot anchor. Such an environment allows for cell-cell interaction without the mechanical influence of a solid surface, which can result in better control of cell phenotype, 3D cell structure uniformity, and stem cell differentiation. This configuration also positions the cells in the 3D cell structure proximal to the culture's source/sink for gas exchange. The short diffusion path length within the liquid allows for higher oxygen flux during metabolic demand. This can decrease the stress on the cells from hypoxia and can mitigate hypoxia induced cell death in the interior of the cell agglomerate. This can also allow for growth of larger spheroids without necrotic cores. Despite the advantages, the use of the Hanging droplet 3D Cell Culture method is not often used due to the complexity of preparation and maintenance of the cultures, as well as a lack of scalability.

Among the coatings that can be used for culturing spheroids, superhydrophobic materials exhibit water contact angles greater than 150° and sliding angles below 10°, causing water to bead up and roll off easily. These surfaces mimic natural examples such as lotus leaves and butterfly wings, where micro- and nanoscale textures, combined with low-surface-energy chemistry, lead to extreme water repellency [10, 11]. Technologies for creating superhydrophobic surfaces have been extensively developed and can be grouped into the following categories: (1) top-down micro/nano fabrication, such as photolithography and etching, which are used to create controlled surface roughness for silicon and polymer substrates [12]; (2) laser ablation, which utilizes femtosecond and nanosecond lasers to directly sculpt hierarchical textures on metals, ceramics, or polymers [13]; (4) bottom-up fabrication, including chemical vapor deposition (CVD), which deposits thin films of low-surface-energy materials, like perfluorinated silanes over nanostructures [14], and electrochemical deposition, which fabricates rough metallic or oxide coatings with high water repellency [15]; (3) solution-based coatings, including spray, dip, or spin coating, which often uses silica or titania nanoparticles in combination with hydrophobic agents like stearic acid, fluoropolymers, or silanes [16], and polymer blends and phase separation, used for phase-separated polymeric films to generate nano/microtexture [17]; (4) plasma treatments and surface modification, including oxygen/argon plasma etching, which modifies surface roughness and energy on substrates like PET, nylon, or polystyrene [18], and plasma polymerization, which deposits ultrathin hydrophobic coatings like PTFE or fluorocarbons [19]; (5) template-based replication, in which natural or engineered microtextures (e.g., lotus leaves, silicon masters) are replicated into materials like PDMS or thermoplastics [20]. Common material systems employed [21] include a structure provider, such as, silica/TiO₂/ZnO NPs, PDMS/PU/PMMA, or CNTs/graphene oxide, and a corresponding surface chemistry modifier, such as fluorinated silanes (PFOTS, PFTMS), stearic acid, alkylsilanes, or PDMS blends, fluorinated polymers. These materials have found applications in a wide array of areas that have several applications, including self-cleaning surfaces [22], corrosion resistance [23], anti-icing, which delays ice nucleation and minimizes adhesion, and biomedical interfaces [24], oil/water separation [25], drag reduction, in which trapped air cushions reduce friction in microfluidics or underwater vehicles [26], and biomedical interfaces, acting as protein-repellent, and anti-fouling, or hemocompatible coatings for implants and sensors [27]. Superhydrophobic materials, formed via a variety of physical, chemical, and hybrid techniques, have rapidly evolved from biomimetic curiosities into practical materials used across engineering, biomedical, environmental, and consumer applications.

Current technology for culturing spheroids provides very limited control over the culture microenvironment, with respect to mass transfer. When spheroids are cultured resting on the bottom of a culture vessel they are under stress from these diffusion limitations, over the distance of the media height, and the distance to the center of the spheroid. Additionally, diffusion of soluble species near a solid surface is limited by the boundary layer effect. The proximity of the spheroid to the bottom of the vessel places it within the boundary layer where diffusion is slower and limits the spheroid's access to nutrients in the media [28]. Accordingly, there is a significant need for improvement in the field of culturing spheroids.

### BRIEF SUMMARY OF THE INVENTION

The subject invention pertains to a novel gas exchanger system and methods thereof for culturing spheroids, organoids, surface attached cultures, and suspension cultures, comprising a substrate 100 having an exterior surface 110, an interior surface 125, and an inter-layer 120, where the inter-layer 120 forms channels 150 that remain unwetted during submersion in a liquid, for example, culture media, allowing continuity between gas volumes when submerged, where the interior surface 125 adheres to a flat bottom multi-well plate 130 forming a structural layer, where the exterior layer comprises a superhydrophobic material and a plurality of surface features 140 decorating the exterior layer of the structure. In embodiments, the substrate 100 comprises hierarchical high aspect ratio surface features 140 including small dimple indentations in the surface and smaller vents 160 that penetrate from the exterior surface 110 of the substrate 100 to the inter-layer 120 where the channels 150 connect the vents 160 to the exterior surface, and thereby the dimple indentations, allowing the exchange of gasses. In embodiments, the surface features 140 include, but are not limited to, shapes, such as cylinder or rounded cone. In embodiments, more generally the surface features 140 can have platonic shapes, optionally with rounded edges.

In preferred embodiments, the superhydrophobic material coating the exterior surface 110 of the substrate 100 creates a dynamic air-liquid interface 190 from surface tension that supports the culture medium's volume and guides cells to form spheroids. In embodiments, the dynamic air-liquid interface 190 is in continuous connection and/or communication with the channels 150, where the channels 150 transport air. In embodiments, the interface extends from and is continuous with the surface of the overlay medium and serves as a gas exchange source without slowing down nutrient diffusion.

In embodiments, the dynamic air-liquid interface 190 comprises a stable air layer 170 in diffusive contact with an overlaying atmosphere 310 allowing diffusive transport of gas components to occur without interruption along the entire length of the stable air layer.

In embodiments, cells seeded over the substrate 100 guided by the dynamic air-liquid interface 190 from the surface tension grow as spheroids 400, where the position of each spheroid 400 is limited to a concavity or dimple. In preferred embodiments, a spheroid 400 floats within the space created by the concavity.

### BRIEF DESCRIPTION OF THE DRAWINGS

FIGS. 1A-1D shows a diagram of the process of cell attachment to a cell culture surface. FIG. 1A shows the cell before adhesion not in contact with the culture vessel. FIG. 1B shows first contact and initial adhesion of the cell to the vessel surface. FIG. 1C shows the cell forming bonds and flattening to the culture surface. FIG. 1D shows the cell after completing the attachment and spreading to conform to the vessel surface.

FIG. 2 shows a diagram of hanging droplet 3D cell culture.

FIG. 3 shows a micrograph of StemCell Technologies' AggreWell 400 Microwell Plate showing the inverted pyramid microfeature and cultured cell clusters at the centers.

FIG. 4 shows a micrograph of Corning Elplasia plates (left) round bottom type plates with Corning Ultra-Low Attachment (ULA) surface, (right) Corning Elplasia square bottom type plates, both with cell spheroids.

FIGS. 5A-5C illustrate three examples of spheroid formation techniques. FIG. 5A shows cells suspended in droplets attached to hanging drop plates. FIG. 5B shows Ultra-low attachment (ULA) plates that inhibit cells from adhering to the surface of the wells forcing them to aggregate and form spheroids. FIG. 5C shows a Spinner Flask agitating a suspension of spheroids to keep them in suspension.

FIGS. 6A-6C show cells growing in a hydrogel. FIG. 6A shows single cells distributed in a hydrogel after cell seeding. FIG. 6B shows small groups of cells that formed from the single cells. FIG. 6C shows spheroids formed from the groups of cells in the same positions as the single cells in FIG. 5A.

FIGS. 7A-7D illustrate the proposed hierarchical structure of the device with sub millimeter sized dimple and spheroid containment. FIG. 7A shows the liquid air boundary interaction at the nanometer scale. FIG. 7B shows the liquid air boundary interaction at the micron scale. FIG. 7C shows the liquid air boundary at the millimeter scale and FIG. 7D shows the vents and channel beneath the outer surface that provides connection between pockets of air layer in the dimples.

FIG. 8 shows spheroids of different sizes in a dimple being laterally confined and supported by the air layer.

FIGS. 9A-9F show a comparison of the oxygen concentration gradient formed by a spheroid growing in a microwell geometry with a traditional solid bottom device and one with a float layer device. FIG. 9A shows the location of the spheroid in the traditional setup with the spheroid in a microwell beneath a depth of growth media in a culture vessel. FIG. 9B shows the simulated oxygen concentration gradient formed during growth of a spheroid in a traditional setup. The oxygen concentration near the spheroid is low and increases in the direction away from the bottom of the vessel. FIG. 9C shows a closeup of the spheroid positioned in the microwell of the traditional setup. The microwell dimension is twice the spheroid dimension. The spheroid lies on the bottom surface of the microwell. FIG. 9D shows the location of the spheroid in the float layer device setup with the spheroid held in a curvature of surface tension formed by the dimple and beneath a depth of growth media in a culture vessel. FIG. 9E shows the simulated concentration gradient formed during growth of a spheroid in a float layer device of the subject invention. The oxygen concentration near the spheroid is high in the direction of the dimple surface and dips very little in the direction of the overhead surface of the growth media. FIG. 9F shows a closeup of the spheroid positioned in the microwell of the Float layer setup of the subject invention. The dimple dimension is twice the spheroid dimension. The spheroid lies on the surface tension above the dimple. The gas layer above the dimple is in contact with the gas volume in the channel below the dimple.

FIGS. 10A-10C show a very simple design that is capable of forming a float layer. FIG. 10A shows a pillar with a triangular cross section that has channels down each of the sides of the triangle. The arrows point to a channel on one face of the pillar and the contact surface at the point of the triangle, which supports the span of the surface tension across the channel. FIG. 10B shows a cross section of the pillar with arrows pointing to a geometric cut into one side of the triangle that forms a channel on the pillar and a point of the triangle that forms the contact surface on the pillar. FIG. 10C shows the cross section of the pillar with a dotted line, which represents the liquid gas interface that forms from the interaction of the surface tension with the superhydrophobic surface of the pillar. The interface contours closely the points of the triangle that form the contact surface, and forms a circular curved surface within the cavity of the shape that forms the channel. The cross sectional area of the gas phase near the surface of the channel is much greater than the cross sectional area of the gas phase near the surface of the points. The curved surface tension within the cavity is not supported by the underlying surface and its shape is not perturbed from circular in that region.

FIGS. 11(i)-11(iv). FIG. 11(i) shows a cad rendering of the base of a prototype float layer device with multiple hexagonal shaped dimples and centering prongs around them. FIG. 11(ii) shows a prototype that has been 3D printed and coated with superhydrophobic coating, containing cell culture media with cells, with a magnified portion of the photo pointing out the reflection of the plastron formed between ridges. FIG. 11(iii) shows a micrograph of the cell culture in the prototype pre-aggregation without any cells having accumulated near the plastron. FIG. 11(iv) shows a micrograph of the cell culture in the prototype post-aggregation with an accumulation of cells near each plastron reflection.

FIG. 12 shows a fluorescence micrograph of a distribution of spheroids grown in the design prototype float layer device from HT-29 colorectal cancer cells. Live cells are stained in green (calcien AM) and dead cells are stained in red (Ethidium-III). All spheroids show substantial green staining and only the larger spheroids show red staining at their centers.

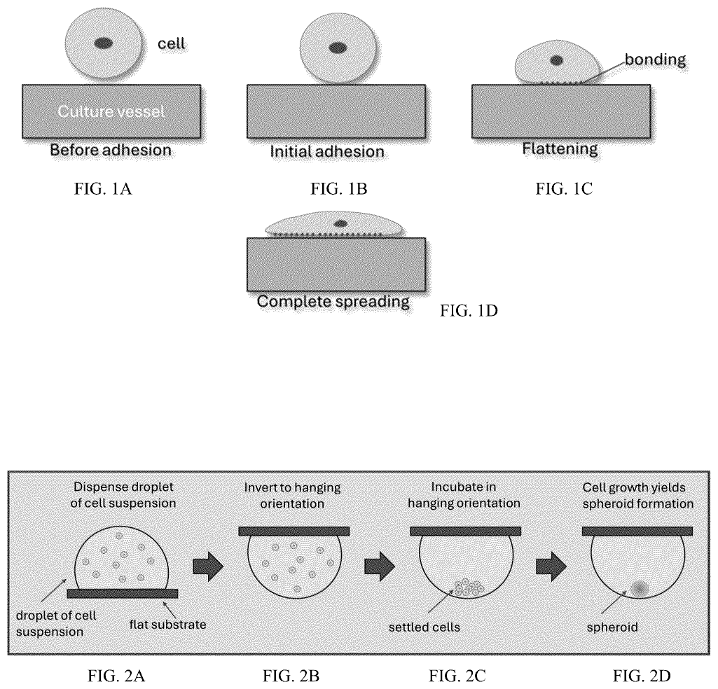

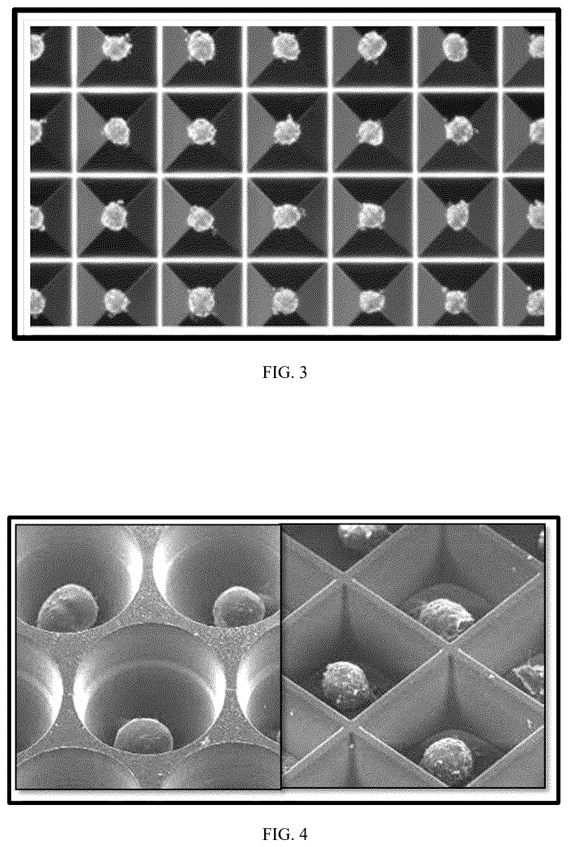

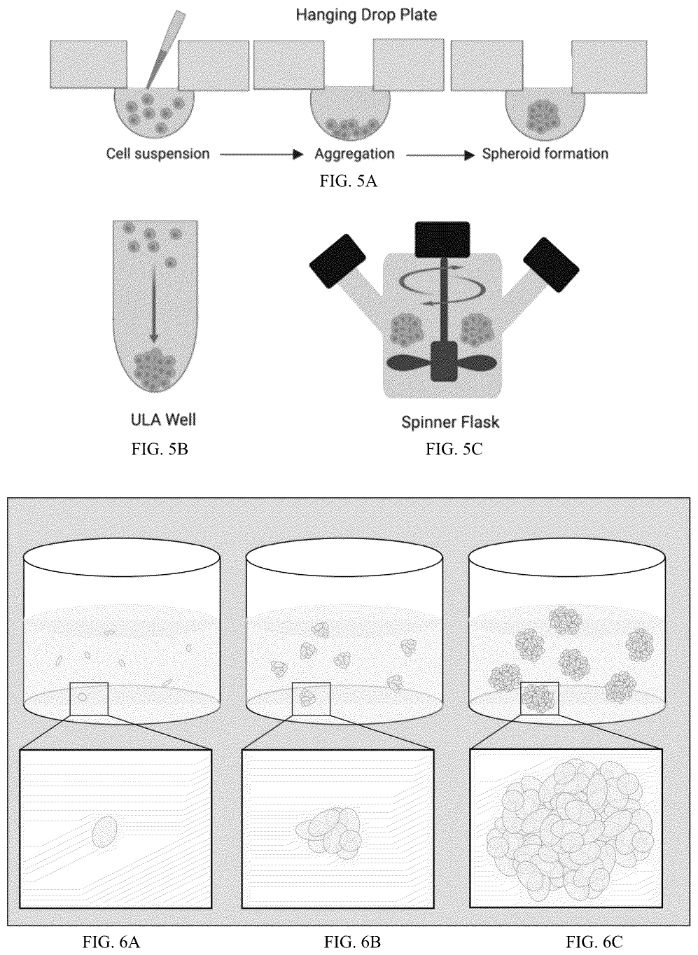

### DETAILED DISCLOSURE OF THE INVENTION

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the term “about” is used, these compositions contain the stated amount of the ingredient with a variation (error range) of 0-10% around the value (X±10%). In other contexts, the term “about” is providing a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

As used herein, the terms “gas-liquid interface” and “air-liquid interface” are used interchangeably.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

As used herein, the terms “determining,” “measuring,” and “assessing,” and “assaying” are used interchangeably and include both quantitative and qualitative determinations.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims.

All references cited herein are hereby incorporated by reference in their entirety.

### Description and Practical Uses of the Invention

For the most part, cells in an organism are not floating around as single cells or required to grow on an inert surface as a monolayer, but are found in a volume surrounded by extracellular matrix interstitial fluid and other cells. There have been several successful techniques and technologies that approximate this environment to varying degrees.

The subject invention pertains to a system and methods for culturing spheroids. In embodiments, the subject invention discloses a novel technology for culturing spheroids, named “Float Layer Cell Culture”, that replaces low-attachment surface coatings on traditional spheroid culture vessels. The invention utilizes hierarchical high aspect ratio surface features 140 to culture spheroids above a liquid-gas boundary. This removes the concern from seeding to the interaction of cells with a ridged non-dynamic surface, which is required in most other 3D culture systems, and increases the flux rate of gasses to and from the 3D culture. In embodiments, the Float Layer Cell Culture system reduces hypoxic stress on the cells and decreases necrotic core formation due to lack of oxygen.

In embodiments, the subject invention utilizes a hierarchical structure from a nanometer to micrometer range, which represents the minimal requirement for the emergence of a superhydrophobic behavior, into the millimeter range, which provides an additional behavior, i.e., the exclusion of liquids from a volumetric layer (“volume exclusion”) having a surface contacting the surface geometry of the hierarchical structure and air-liquid interfaces formed by surface tension spanning between the surface geometries, which can be utilized in a submerged structure to generate functional mechanical surfaces, formed by the surface tension, that dynamically interact with cells and gas exchange volumes in close contact with the liquid volume that provides a source/sink for dissolved gasses in a cell culture. In embodiments, to improve culture environment control, the bottom of the culture device is replaced with an engineered surface where the surface geometry incorporates small dimple indentations in the surface and smaller vent holes or channels 150 that penetrate through the surface. This surface geometry is coated with a superhydrophobic low energy surface treatment that inhibits surface wetting. In preferred embodiments, the superhydrophobic surface extends the air-liquid boundary across a much larger surface permitting a density of spheroid 400 forming feature per culture area and a shared continuous liquid volume. In more preferred embodiments, when culture media is added, an air layer forms across the bottom surface of the vessel and remains during culturing. This air layer conforms to the shape of the small indentations while being in continuous communication with the smaller vents 160, where the vents 160 are in continuous communication with the channels 150. In more preferred embodiments, this configuration produces a dynamic air-liquid interface 190 from surface tension that supports the culture medium's volume and guides cells to form spheroids. This interface serves as an oxygen source and does not slow down nutrient diffusion, giving better control over the culture environment.

In embodiments, the float layer interface puts the oxygen source immediately adjacent to the microenvironment, removing its dependence on diffusion over large distances, i.e., from the air diffusing from the surface of the growth medium. Replacing the solid surface with dynamic air-liquid surface tension eliminates the boundary layer associated with a solid surface and allows for better movement of soluble nutrients, thus providing more direct, predictable control of the spheroid culture environment, yielding more reproducible models for research.

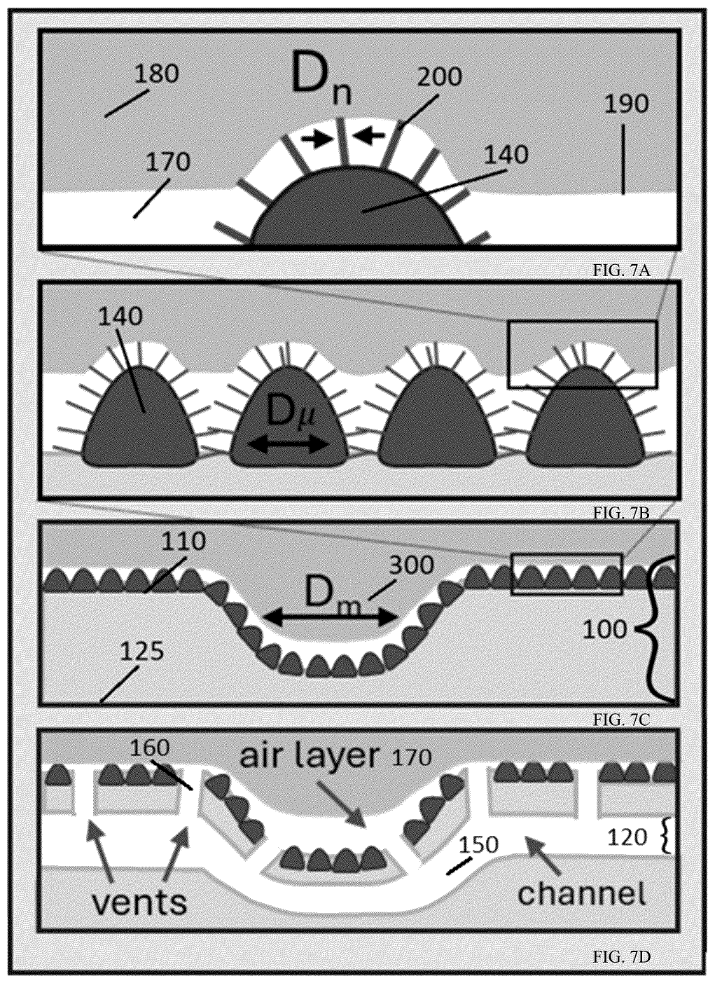

In embodiments, the thin blue lines and the rounded cones shown in FIGS. 7A and 7B represent the surface coating allowing the surface to create a superhydrophobic behavior generated by two levels of hierarchy, i.e., nanometer sized hydrophobic particles, including, but not limited to, small channels 200, ranging from about 5 to about 20 nm, contacting the surface of micron sized hydrophobic particles, ranging from about 1 to about 10 μm, arranged on a flat hydrophobic surface. In embodiments, this hierarchical structure produces a greater than 150 degrees contact angle and superhydrophobic hysteresis of droplets. In preferred embodiments, the subject invention extends the hierarchical structure into the millimeter range, where additional behaviors, i.e., liquid volume exclusion and surface tension spanning structures that can be utilized in a submerged structure to generate functional mechanical surfaces formed from surface tension and gas exchange volumes in close contact with the liquid volume.

In embodiments, the small channels 200 have a diameter in the nanoscale (Dₙ), the surface features 140 have a size in the micrometer scale (Du), and up to the millimeter scale, and the concavities are in the millimeter scale (Dₘ) (FIG. 7).

In preferred embodiments, the thin blue lines (FIG. 7A-7C) are small channels 200 or particles on the nanometer scale that represent the lowest level of the hierarchical surface structure or roughness that enables superhydrophobic behavior.

In embodiments, the float layer air volume within a dimple is in gas phase volumetric contact with the air volume in other dimples, as well as with an air reservoir or with atmosphere (FIG. 7).

In preferred embodiments, the air-liquid interface 190 creates a surface tension support layer that keeps a spheroid 400 floating within a dimple and provides oxygen directly to the surface of the spheroid, thus optimizing diffusive flux. This dynamic support layer eliminates the boundary layer associated with solid surfaces and improves lateral diffusion of nutrients and metabolites between the cell formation and the culture medium. With improved control over oxygen and nutrient availability, spheroids can be grown with greater reproducibility resulting in more accurate and reliable in vitro models.

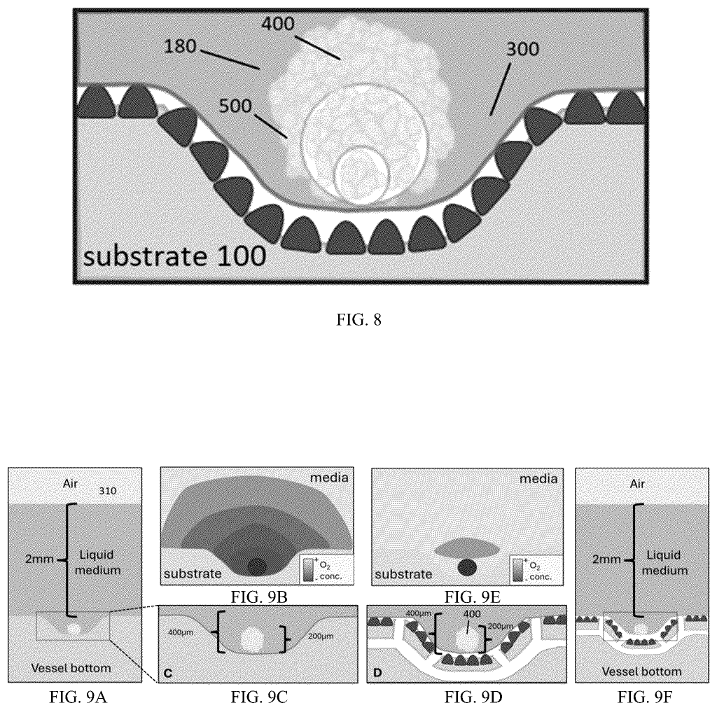

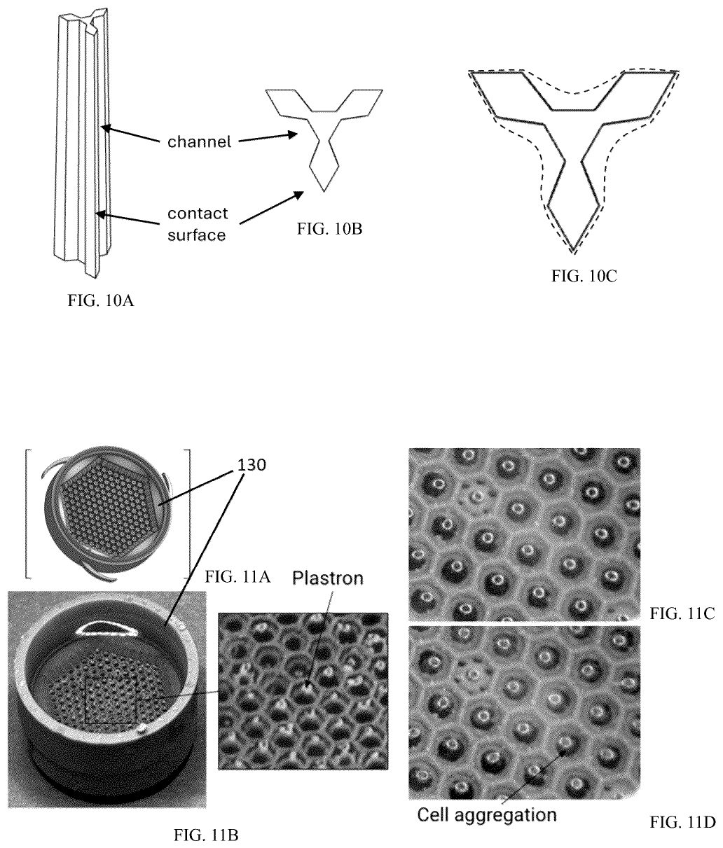

Increasing the surface area of the air-liquid interface 190 is routinely achieved in nature and in the lab. However, without further control of the surface geometry on the millimeter/sub-millimeter scale, cells settle and clump randomly on a flat surface. While cells need vertical support provided with a fluid-fluid surface tension boundary, they also need lateral confinement. In embodiments, incorporating sub millimeter concave features, or dimples, into the surface configuration allows for a droplet like geometry, thus providing a mechanism for the cells to collect at a central position and lateral confinement of the individual spheroids while maintaining separation between spheroids once formed (FIG. 8).

In certain embodiments, the dimple geometry utilized in the subject invention acts as a funnel when the cells are settling, collecting at the bottom of the concave shape of the dimple. After settling, the cells form a spheroid 400. Then, as the spheroid 400 grows, it remains within the dimple due to gravity. This keeps individual spheroids 400 separated while sharing the same main volume of growth medium. In preferred embodiments, medium exchanges can be performed by replacing most of the overlying volume without disturbing the settled spheroids 400.

In embodiments, a spheroid 400 floating in a concavity or dimple is not in continuous contact with the surface of the substrate 100. Occasional contact may happen, especially during media exchange.

In certain embodiments, the configuration described above offers an improvement on current technologies by allowing the self-assembly of the seed cell clusters without the need to rely on extra cellular matrix (ECM) contractile forces after the dispersed seeding of cells on a surface, or centrifugation to force a central collection of cells.

In preferred embodiments, the advantage of creating an air-liquid interface 190 directly adjacent to the spheroid 400 volume is providing a higher oxygen flux to the growing spheroid 400 (FIG. 7). In a configuration where the area of the air-liquid interface 190 is expanded, the diffusion distance to the spheroid 400 within the liquid is very short, but the distance the oxygen must diffuse within the gas layer to get to a spheroid 400 in the center of the area increases by the square root of the area. In a continuous hydrophobic surface with a liquid pancake overlay, oxygen can only diffuse in from the edge of the liquid layer and through the height of the liquid layer, and is limited by the cross sectional area of the gas volume orthogonal to the shortest path of diffusion. In embodiments, to take advantage of improved oxygen flux, the surface configuration decreases the “in gas” diffusion distance to a convectively mixed volume of air (FIG. 8). In embodiments, this applies also to the flux of carbon dioxide away from the spheroid 400 and the maintenance of pH.

In preferred embodiments, access channels 150 are provided through the superhydrophobic substrate 100 to decrease the distance from the air-liquid interface 190 to the bulk atmosphere. Once adequate access to the bulk atmosphere is provided to the float layer, the primary source of oxygen for cellular metabolism is no longer the air diffusing from the surface of the growth medium.

In certain embodiments, the culture system can provide oxygen at a rate comparable to in vivo conditions. In preferred embodiments, the system is scalable to high volumes with high throughput.

In some examples, the height of the growth medium volume is not a limiting factor in cell growth kinetics. This increases the reproducibility between protocols that have different standard media volume requirements. Accordingly, the length of time between media exchange for feeding the culture can be decreased and a greater volume of media can be present per surface area at any time.

In certain embodiments, the aqueous solution comprises a hydrogel, where the hydrogel comprises a Corning® Matrigel® or comparable substitute.

In embodiments, the material utilized for creating a superhydrophobic surface with microcavities includes, but is not limited to, polyvinylidene fluoride (PVDF) with microtexturing, polypropylene with nanotexturing, and polydimethylsiloxane (PDMS) with silica nanoparticles.

The above-described system and methods are described herein for the purposes of illustration and are not intended to be limiting. Alternative and additional variations may be apparent to those skilled in the art.

### Materials and Methods

Many materials in nature use a combination of material chemistry and submicron geometry to achieve very low wetting angles. The lotus effect is one example, which was discovered in 1964 by Dettre and Johnson, and refers to the superhydrophobic behavior of lotus leaves to form high surface contact angles with water droplets that allows for self-cleaning of dirt from the leaves.

In practice, when the volume of the water droplet is increased, the air layer between the liquid and the solid surface is maintained. This can be seen when a larger amount of water pools at the center of the lotus leaf. A silvering optical illusion occurs when viewed from some angles. This is due to the liquid gas interface beneath the liquid volume. When going from water to air as optical media, there is a large negative change in refractive index. This results in total internal reflection of some rays of light and looks like a silvering or mirroring effect.

There are several factors that we considered critical design elements for the design of the 3D cell culture device of the subject invention. Thes factors comprise (a) a superhydrophobic contact surface that provides mechanical support of the volume through surface tension between a liquid gas interface, (b) confinement geometry that provides dynamic cell collection at seeding and lateral confinement during spheroid growth, c) air exchange channels that maintain gas mix concentration equilibrium with the environment, (d) rapid design and material strength, because the material and design must withstand mechanical stress, wear, and environmental conditions without degrading, (c) contamination resistance, because the surface should be resistant to contamination from dust, biofilm, or other particles to maintain functionality, (f) chemical stability, because the surface coating must resist chemical degradation from exposure to cell culture media and other chemicals used in the process, (g) thermal stability, because the surface should remain stable and maintain its properties under expected temperature fluctuations, (h) uniformity, because the microcavities must be uniformly distributed and consistent in size to ensure reproducibility and uniform spheroid formation, (i) biocompatibility, because materials used must be biocompatible, ensuring no adverse effects on cell growth and viability, (j) scalability, because the design should allow for scaling up to larger areas to accommodate multiple spheroids and larger culture volumes, (k) cost effectiveness, because the manufacturing process and materials should be cost-effective for practical implementation and widespread use, and (1) user-friendly design, because the device should be easy to set up, operate, and integrate into existing laboratory workflows.

Additive manufacturing in the form of resin LCD 3D printing has been used to prototype the form of multiple designs. Using biocompatible resins this method can produce 3D solids that meet all of the structural and non-surface related constraints mentioned above. Although this method can be refined and validated for mass production of devices, it is not the most cost effective method for mass production. Some designs lend themselves to injection molding or cast molding, both of which are methods capable of producing the required features with sufficient dimensional size and precision to produce forms capable of being processed with a surface coating that meets the additional surface wetting requirements for making a fully functional float layer device.

The low wetting angle surface coating has been applied with multi step dip coating and spray coating. The application sequence is: (1) surface priming, (2) adhesive course texture coating, and (3) top fine texture coating. The final cured materials resulting from resin 3D printing, mostly acrylate variants, require priming to add functional groups that allow for spray coated layers to sufficiently adhere to the surface of the devices. Lye at about 0.5N or about 10% aminopropyl trimethoxysiloxane in dry isopropanol is applied by submersion coating. After removal from the priming solution the work in progress device is allowed to dry at ambient conditions for about 1 h, rinsed with about 99% isopropanol, and cured in a drying oven at 80° C. for 30 min.

The adhesive layer is a commercially available mixture of polyurethane based adhesive silica particles in the range of about 1 um to about 100 nm. It is applied evenly to the surface of the device in either a can or air aerosol mist in two light coats with about 2 min of drying time between the coatings. The surface is allowed to dry for about 30 min at standard conditions before the final coating is applied.

The top coat comprises a commercially available PDMS based material with silica nanoparticles in the range of 10-20 nm with fluorene functionalization. This material is also applied evenly to the device surface via an aerosol applying 3 light coats with about 2 min dry time between coats. Application of the top coat is followed by about 1 h incubation in a drying oven at about 60° C. to fully dry any volatiles and anneal the layers to the surface of the device.

After the device is cooled from the incubation step it is submerged in deionized water and agitated for about 30 s to remove any residual free powder from the top coat step. After removal from the water the device is allowed to dry at standard conditions for 1 h before use or sterilization.

The devices are sterilized using a steam autoclave. Devices are loaded into the autoclave and processed on a dry cycle, 30 min at 121° C. and at a pressure of 15 bar. The devices must be allowed to dry completely before they regain their hydrophobic properties.

### Suitable Material Technologies

Materials that meet the requirement for use in the production of a device having the correct macrostructure, microstructure and surface chemistry, include, but are not limited to:

Polydimethylsiloxane (PDMS) with Silica Nanoparticles: PDMS is widely used in microfabrication for its flexibility and biocompatibility. When combined with silica nanoparticles, it can achieve superhydrophobic properties.

Fluorinated Silanes Coated on Silicon or Glass: Fluorinated silanes can create a superhydrophobic layer when coated on silicon or glass substrates. They provide excellent chemical resistance and stability.

Titanium Dioxide (TiO2) Nanostructures: TiO2 can be structured to create microcavities and, when treated with a hydrophobic coating, can provide superhydrophobicity and resistance to contamination.

Perfluoropolyether (PFPE) Coatings: PFPE is a highly stable and chemically resistant material that can be used to coat microstructure surfaces to achieve superhydrophobicity.

Polypropylene with Nanotexturing: Polypropylene is a chemically resistant polymer that can be nanotextured to create microcavities and achieve superhydrophobic properties.

Graphene Oxide Coated Polymers: Polymers like polycarbonate or polyethylene terephthalate (PET) coated with graphene oxide can provide a combination of superhydrophobicity and mechanical robustness.

Polyvinylidene Fluoride (PVDF) with Microtexturing: PVDF is a durable polymer with excellent chemical resistance. Microtexturing can enhance its hydrophobic properties.

Carbon Nanotube (CNT) Composites: Embedding CNTs in a polymer matrix can create a superhydrophobic surface with microcavities and high chemical stability.

Polyurethane (PU) with Fluorinated Additives: PU is versatile and can be combined with fluorinated additives to enhance its hydrophobic and chemical-resistant properties.

Silicon Dioxide (SiO2) Aerogels: SiO2 aerogels have a highly porous structure that can be modified to create microcavities and coated for superhydrophobicity and chemical resistance.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1

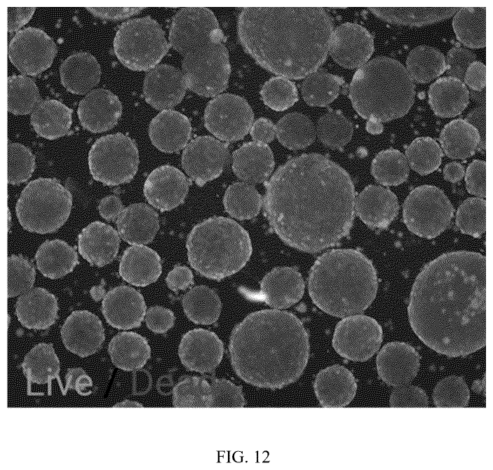

An acrylic 3D print of the prototype (FIG. 11(i)) was printed with a resin 3D printer and coated with the three step coating process to yield a superhydrophobic surface on the device. The device was seeded with 3 mL of 10⁵ cells per mL of HT-29 cells, a colorectal cancer cell line (FIG. 11(ii)) A micrograph of the dimples in the device immediately after seeding showed no cells accumulated near the plastrons (FIG. 11(iii)). After 2 h another micrograph was taken that showed cells had started to accumulate near the plastrons within the dimples of the device (FIG. 11(iv)). The cell culture in the device was incubated for 2 days at 37 C and 5% CO2, as prescribed by the normal culturing protocol for HT-29 cells. After incubation the media and cells were harvested from the device and stained for live/dead fluorescence. A two population distribution of cells was observed (FIG. 12). A distribution of spheroids was observed with a majority of the cells staining green revealing that they were living (FIG. 12). There was an occurrence of red/dead stain but it was only minimal and in the center of larger spheroids (FIG. 12). This demonstrates the ability to cultivate spheroids on a surface tension boundary that is in contact with an atmosphere connected gas volume.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### EXEMPLARY EMBODIMENTS

Embodiment 1. A gas exchanger system for culturing spheroids, the system comprising:
(a) a substrate 100 having an exterior surface 110 an inter-layer 120, and an interior surface 125 wherein the interior surface 125 adheres to a flat bottom multi-well plate 130 forming a monolayer, wherein the exterior surface 110 comprises a superhydrophobic material; and
(b) a plurality of surface features 140 decorating the exterior surface 110 of the substrate 100 wherein a surface feature 140 is optionally shaped as cylinder or cone, wherein the surface features 140 have a diameter ranging from about 100 microns to about 1 millimeter;
(c) a plurality of channels 150, wherein the channels 150 are in the inter-layer 120, wherein each channel 150 is in continuous communication with a plurality of vents 160, wherein the channels 150 remain unwetted during submersion in a liquid,
wherein each vent 160 is in continuous communication with the exterior surface 110,
wherein the exterior surface 110 is overlaid by a stable air layer 170, wherein the stable air layer 170 is in continuous communication with the plurality of vents 160, wherein the vents 160 allow the passage of air and/or oxygen from the channels 150 to the stable air layer,
wherein an axis of a surface feature 140 is oriented in a direction tangential to the exterior surface 110,
wherein one or more layers of surface features 140 are stacked on the exterior surface 110 of the substrate 110,
wherein a multitude of small channels 200 connect a surface of each surface feature 140 to the exterior surface 110 of the substrate 100, wherein the small channels 200 allow the passage of gases and resist liquid incursion,
wherein a tangent line 210 formed between any part of the surface feature positioned within a distance half the size of the cylinder or rounded cone forms an acute angle with a local normal direction of the exterior surface,
wherein the surface of the surface feature 140 and the exterior surface 110 are functionalized with a hierarchical surface roughness having low energy chemical makeup and a water contact angle greater than 130 degrees,
wherein the exterior surface 110 comprises a multitude of hierarchical structures, wherein the hierarchical structures comprise a multitude of concavities or dimples 300,
wherein the hierarchical structures comprises at least two levels of hierarchy, wherein the first level comprises nanometer sized hydrophobic small channels 200 or particles ranging from about 5 to about 20 nm, contacting the second level comprises a surface of micron sized hydrophobic particles or channels 150, ranging from about 1 to about 10 μm, arranged on a flat hydrophobic surface, wherein a third level of hierarchy comprises the surface features 140,
wherein the stable air layer 170 contacts an aqueous solution 180, forming an air-liquid interface 190, wherein the stable air layer 170 is in diffusive contact with an overlaying atmosphere 310 allowing diffusive transport of gas components to occur without interruption along an entire length of the stable air layer 170,
wherein the system is configured such that a position of a spheroid 400 comprising cultured cells 500 is limited by a surface tension produced by the air-liquid interface 190 to a concavity 300, wherein the spheroid 400 floats within the concavity 300, and
wherein the air-liquid interface 190 is configured to provide oxygen to the cultured cells 500.
Embodiment 2. The system of embodiment 1, wherein the surface feature 140 comprises a platonic shape.
Embodiment 3. The system of embodiment 2, wherein the edges of the platonic shape are rounded.
Embodiment 4. The system of claim 1, wherein the water contact angle is greater than 150 degrees.
Embodiment 5. The system of any preceding embodiments, wherein the gas mixture atmosphere comprises oxygen and nitrogen at a relative concentration of about 21% and about 78%, respectively.
Embodiment 6. The system of any preceding embodiments, wherein the aqueous solution 180 comprises a hydrogel.
Embodiment 7. The system of any preceding embodiments, wherein the substrate 100 and the aqueous solution 180 are contained within a plate, wherein an inner surface of the plate provides solid support for the substrate 100 and the aqueous liquid, wherein the stable air layer 170 contacts the aqueous solution 180 allowing the passage of air through the air-liquid interface 190 from the stable air layer 170 to the aqueous solution.
Embodiment 8. The system of any preceding embodiments, wherein the external surface comprises a superhydrophobic surface comprising hierarchical structures, wherein the material of the superhydrophobic surface comprises polyvinylidene fluoride (PVDF) with microtexturing, polypropylene with nanotexturing, and polydimethylsiloxane (PDMS) with silica nanoparticles.

### REFERENCES

1. Carrel, A. ‘On the Permanent Life of Tissues Outside of the Organism.’ Journal of Experimental Medicine, 1912.
2. Freshney, R. I. ‘Culture of Animal Cells: A Manual of Basic Technique.’ Wiley, 2010.
3. Edmondson, R. et al. ‘Three-Dimensional Cell Culture Systems and Their Applications in Drug Discovery and Cell-Based Biosensors.’ Assay Drug Dev Technol, 2014.
4. Sutherland, R. M. ‘Cell and Environment Interactions in Tumor Microregions: The Multicell Spheroid Model.’ Science, 1988.
5. Corning Life Sciences. ‘Elplasia Plates for 3D Cell Culture,’ Technical Bulletin CLS-CC-073.
6. Hsiao, A. Y. et al. ‘Micro-ring Structures Stabilize Microtissues for High-Throughput Screening.’ Biotechnol Bioeng, 2012.
7. Fukuyama, T. et al. ‘Development of a Hanging-Drop 3D Culture System for Human Hepatocytes.’ Sci Rep, 2015.
8. Griffith, L. G. & Swartz, M. A. ‘Capturing Complex 3D Tissue Physiology In Vitro.’ Nat Rev Mol Cell Biol, 2006.
9. Jain, R. K. ‘Molecular Regulation of Vessel Maturation.’ Nat Med, 2001., 2010.
10. Barthlott, W., & Neinhuis, C. (1997). Purity of the sacred lotus, or escape from contamination in biological surfaces. Planta, 202 (1), 1-8.
11. Nosonovsky, M., & Bhushan, B. (2008). Multiscale effects and capillarity in bioinspired surfaces. Microsystem Technologies, 14 (10), 1409-1420.
12. Li, X.-M., Reinhoudt, D., & Crego-Calama, M. (2007). What do we need for a superhydrophobic surface? A review on the recent progress in the preparation of superhydrophobic surfaces. Chemical Society Reviews, 36 (8), 1350-1368.
13. Long, J., Zhong, M., Zhang, H., & Fan, P. (2015). Superhydrophobic surfaces fabricated by femtosecond laser with tunable water adhesion: From Lotus leaf to rose petal. ACS Applied Materials & Interfaces, 7 (18), 9858-9865.
14. Shirtcliffe, N. J., McHale, G., Newton, M. I., Perry, C. C., & Roach, P. (2005). Porous materials show superhydrophobic to superhydrophilic transitions. Chemical Communications, (25), 3135-3137.
15. Liu, T., Sun, T., Chen, Z., Feng, L., & Jiang, L. (2005). Electrochemical control of the wettability of ZnO nanorod films. Angewandte Chemie International Edition, 44 (30), 5188-5191.
16. Darmanin, T., & Guittard, F. (2015). Recent advances in the design of epoxy-based superhydrophobic surfaces: From chemistry to topology. Chemical Reviews, 115 (14), 6361-6420.
17. Zhang, L., et al. (2008). Superhydrophobic surfaces: From structural control to functional application. Journal of Materials Chemistry, 18 (6), 621-633.
18. Morra, M. (2000). On the molecular basis of fouling resistance. Journal of Biomaterials Science, Polymer Edition, 11 (6), 547-569.
19. Yasuda, H. (2005). Plasma polymerization. Academic Press.
20. Feng, L., Zhang, Y., Xi, J., Zhu, Y., Wang, N., Xia, F., & Jiang, L. (2008). Petal effect: A superhydrophobic state with high adhesive force. Langmuir, 24 (8), 4114-4119.
21. Gao, X., & Jiang, L. (2004). Water-repellent legs of water striders. Nature, 432 (7013), 36.
22. Bhushan, B., & Jung, Y. C. (2011). Natural and biomimetic artificial surfaces for superhydrophobicity, self-cleaning, low adhesion, and drag reduction. Progress in Materials Science, 56 (1), 1-108.
23. Wang, S., Liu, K., Yao, X., & Jiang, L. (2015). Bioinspired surfaces with superwettability: New insight on theory, design, and applications. Chemical Reviews, 115 (16), 8230-8293.
24. Meuler, A. J., Mckinley, G. H., & Cohen, R. E. (2010). Exploiting topographical texture to impart icephobicity. ACS Nano, 4 (12), 7048-7052.
25. Xue, Z., Wang, S., Lin, L., Chen, L., Liu, M., Feng, L., & Jiang, L. (2011). A novel superhydrophilic and underwater superoleophobic hydrogel-coated mesh for oil/water separation. Advanced Materials, 23 (37), 4270-4273.
26. Daniello, R. J., Waterhouse, N. E., & Rothstein, J. P. (2009). Drag reduction in turbulent flows over superhydrophobic surfaces. Physics of Fluids, 21 (8), 085103.
27. Epstein, A. K., Wong, T.-S., Belisle, R. A., Boggs, E. M., & Aizenberg, J. (2012). Liquid-infused structured surfaces with exceptional anti-biofouling performance. PNAS, 109 (33), 13182-13187.
28. Hirschhaeuser, F. et al. ‘Multicellular Tumor Spheroids: An Underestimated Tool Is Catching Up Again.’ J Biotechnol, 2010.

We claim:

1. A gas exchanger system for culturing spheroids, the system comprising:
(a) a substrate 100 having an exterior surface 110 an inter-layer 120, and an interior surface 125 wherein the interior surface 125 adheres to a flat bottom multi-well plate 130 forming a monolayer, wherein the exterior surface 110 comprises a superhydrophobic material; and
(b) a plurality of surface features 140 decorating the exterior surface 110 of the substrate 100 wherein a surface feature 140 is optionally shaped as cylinder or cone, wherein the surface features 140 have a diameter ranging from about 100 microns to about 1 millimeter;
(c) a plurality of channels 150, wherein the channels 150 are in the inter-layer 120, wherein each channel 150 is in continuous communication with a plurality of vents 160, wherein the channels 150 remain unwetted during submersion in a liquid,
wherein each vent 160 is in continuous communication with the exterior surface 110,
wherein the exterior surface 110 is overlaid by a stable air layer 170, wherein the stable air layer 170 is in continuous communication with the plurality of vents 160, wherein the vents 160 allow the passage of air and/or oxygen from the channels 150 to the stable air layer,
wherein an axis of a surface feature 140 is oriented in a direction tangential to the exterior surface 110,
wherein one or more layers of surface features 140 are stacked on the exterior surface 110 of the substrate 110,
wherein a multitude of small channels 200 connect a surface of each surface feature 140 to the exterior surface 110 of the substrate 100, wherein the small channels 200 allow the passage of gases and resist liquid incursion,
wherein a tangent line 210 formed between any part of the surface feature positioned within a distance half the size of the cylinder or rounded cone forms an acute angle with a local normal direction of the exterior surface,
wherein the surface of the surface feature 140 and the exterior surface 110 are functionalized with a hierarchical surface roughness having low energy chemical makeup and a water contact angle greater than 130 degrees,
wherein the exterior surface 110 comprises a multitude of hierarchical structures, wherein the hierarchical structures comprise a multitude of concavities or dimples 300,
wherein the hierarchical structures comprises at least two levels of hierarchy, wherein the first level comprises nanometer sized hydrophobic small channels 200 or particles ranging from about 5 to about 20 nm, contacting the second level comprises a surface of micron sized hydrophobic particles or channels 150, ranging from about 1 to about 10 μm, arranged on a flat hydrophobic surface, wherein a third level of hierarchy comprises the surface features 140,
wherein the stable air layer 170 contacts an aqueous solution 180, forming an air-liquid interface 190, wherein the stable air layer 170 is in diffusive contact with an overlaying atmosphere 310 allowing diffusive transport of gas components to occur without interruption along an entire length of the stable air layer 170,
wherein the system is configured such that a position of a spheroid 400 comprising cultured cells 500 is limited by a surface tension produced by the air-liquid interface 190 to a concavity 300, wherein the spheroid 400 floats within the concavity 300, and
wherein the air-liquid interface 190 is configured to provide oxygen to the cultured cells 500.

2. The system of claim 1, wherein the surface feature 140 comprises a platonic shape.

3. The system of claim 2, wherein the edges of the platonic shape are rounded.

4. The system of claim 1, wherein the water contact angle is greater than 150 degrees.

5. The system of claim 1, wherein the gas mixture atmosphere comprises oxygen and nitrogen at a relative concentration of about 21% and about 78%, respectively.

6. The system of claim 1, wherein the aqueous solution 180 comprises a hydrogel.

7. The system of claim 1, wherein the substrate 100 and the aqueous solution 180 are contained within a plate, wherein an inner surface of the plate provides solid support for the substrate 100 and the aqueous liquid, wherein the stable air layer 170 contacts the aqueous solution 180 allowing the passage of air through the air-liquid interface 190 from the stable air layer 170 to the aqueous solution.

8. The system of claim 1, wherein the external surface comprises a superhydrophobic surface comprising hierarchical structures, wherein the material of the superhydrophobic surface comprises polyvinylidene fluoride (PVDF) with microtexturing, polypropylene with nanotexturing, and polydimethylsiloxane (PDMS) with silica nanoparticles.
